(19) United States
(12) Patent Application Publication
(10) Pub. No.: US 2022/0387004 A1
(43) Pub. Date: Dec. 8, 2022

(54) DEVICE FOR COLLECTION OF BIOLOGICAL SAMPLES

(71) Applicant: GRAPHENE-DX, INC., Boston, MA (US)

(72) Inventors: Tom Ruby, Boston, MA (US); Namal Nawana, Weston, MA (US); Kyle Bulloch, Rancho Santa Fe, CA (US); Gerard Vurens, Palo Alto, CA (US); Ian Lang, Santa Clara, CA (US)

(21) Appl. No.: 17/751,364

(22) Filed: May 23, 2022

Related U.S. Application Data
(60) Provisional application No. 63/192,227, filed on May 24, 2021.

(51) Int. Cl. A61B 10/00

(52) U.S. Cl. CPC A61B 10/0051; A61B 2560/0406; A61B 2560/0462; A61B 2560/0487; A61B 2560/0271

(57) ABSTRACT

In one aspect, a sample collection device for collecting a biological sample is disclosed, which comprises a sample-receiving cup having a body that extends from a proximal opening to a distal opening, where the proximal opening is configured for receiving a biological sample. The sample collection device further includes a sensor comprising at least one sensing unit, where the sensor is sealingly connected to the distal opening of the sample-receiving cup such that the sensing unit is exposed to the distal opening. A reservoir for storing one or more sample-processing reagents is provided in the fluid-receiving cup such that the sensing unit is in fluid contact with the reagent(s) stored in the reservoir. A frangible membrane separates the reservoir from the proximal opening.

### CROSS-REFERENCE TO RELATED APPLICATIONS

This application claims priority to U.S. Prov. App. No. 63/192,227, filed on May 24, 2021, and entitled “Device for Collection Of Biological Samples,” which is incorporated herein by reference.

### BACKGROUND

The present disclosure is directed to a system for collecting a biological sample and optionally analyzing the collected sample for determining whether a target analyte is present in the sample.

Biological samples provide important information about a variety of physiological conditions. For example, saliva testing is a diagnostic technique that can be used to identify a variety of markers, such as markers of endocrine, immunologic, inflammatory and infectious conditions, among others. Further, due to its ease of collection, saliva testing can be employed for chronological assessment of such markers.

In addition, there is a need for rapid testing of collected biological samples. The recent COVID-19 pandemic has further accentuated the need for easy, non-invasive, and safe collection of biological samples and providing rapid analysis of such samples, e.g., to identify one or more pathogens in the collected sample.

Accordingly, there is a need for systems for collecting biological samples as well as for systems that allow rapid testing of the collected samples.

### SUMMARY

In one aspect, a device for collecting and analyzing a biological sample is disclosed, which comprises a sample-receiving cup having a body that extends from a proximal opening to a distal opening, where the proximal opening is configured for receiving a biological sample. The sample collection device further includes a sensor comprising at least one sensing unit, where the sensor is sealingly connected to the distal opening of the sample-receiving cup such that the sensing unit is exposed to the distal opening. A reservoir for storing one or more sample-processing reagents is provided in the fluid-receiving cup such that the sensing unit is in fluid contact with the reagent(s) stored in the reservoir. A frangible membrane separates the reservoir from the proximal opening of the sample-receiving cup.

The device further includes a shaft that is configured for introduction into the fluid-receiving cup for piercing the frangible membrane, thereby releasing the sample-processing reagent(s) for mixing with the received biological sample. In some embodiments, the shaft is connected to a cap such that upon insertion of the shaft into the sample-receiving cup, the cap covers the proximal opening of the sample-receiving cup. In some embodiments, the device further includes a housing defining an enclosure in which said sample-receiving cup can be positioned. In some such embodiments, the cap can include a peripheral edge that can engage with the rim of the proximal opening of the sample-receiving cup. In some embodiments, the sample-receiving cup has a funnel-shaped profile such that said proximal opening has a larger area than that of the distal opening. In some embodiments, the device further includes a seal formed between the sensor and the distal opening of the fluid-receiving cup. The seal can be, for example, a gasket.

At least a portion of the processed biological sample can then come into contact with the sensing unit through the distal opening of the fluid-receiving cup. The sensing unit can generate a detection signal when a target of interest is present in the processed biological sample. The target of interest can be, for example, one or more protein, RNA and/or DNA segments.

In some embodiments, the proximal and the distal openings of the sample-receiving cup can be substantially parallel relative to each other, whereas in other embodiments, the proximal and the distal openings can be positioned at an angle relative to one another. For example, in some such embodiments, the distal opening can be formed so as to be substantially orthogonal to the proximal opening. Further, the proximal and the distal openings can have a variety of different shapes. In general, the proximal opening can be configured to facilitate collection of a biological sample of interest, e.g., a saliva sample, and the distal opening can be configured to facilitate the interaction of any of the sample-processing reagents and/or the processed sample with the sensing unit.

In some embodiments, the biological sample can be a subject's saliva. In other embodiments, the biological sample can include a subject's nasal and/or nasopharyngeal swab, urine, blood (including whole blood, serum, and/or plasma), stool, spinal fluid, and/or any other liquid solution taken or derived from the subject.

A variety of sample-processing reagents can be employed in the practice of the present teachings. Some examples of such sample-processing reagents include, without limitation, enzymes and/or any reagent capable of disrupting or lysing cell membranes, e.g., Tris-HCL (hydroxymethyl aminomethane hydrochloride), EDTA (ethylenediaminetetraacetic acid), NP-40 (nonyl phenoxypolyethoxylethanol or Tergitol-type NP-40), Triton (polyethylene glycol tert-octylphenyl ether), etc.

In some embodiments, the sample collection/analysis device can further include a housing that defines an enclosure configured for receiving the sample-collection cup. The enclosure can include a proximal opening through which the sample-collection cup can be inserted into the enclosure. In some embodiments, the enclosure and the sample-collection cup are configured such that upon insertion of the sample-collection cup into the enclosure, the proximal openings of the enclosure and the sample-collection cup are substantially aligned.

The sample-collection cup and the housing can be formed of a variety of polymeric materials. Some examples of suitable polymeric materials include any of PDMS (polydimethylsiloxane), polypropylene, polycarbonate, ABS (acrylonitrile butadiene styrene), HIPS (high impact polystyrene), and PMMA (poly(methyl methacrylate) or acrylic).

In some embodiments, the sample collection/analysis device can include electronic circuitry positioned in the housing and in communication with the sensor for receiving one or more signals generated by the sensor in response to exposure of the sensor to the biological sample. In some embodiments, the sample collection/analysis device includes an interface for communicating sensor output to a user. The interface can include selectively illuminated indicators on an exterior surface of the device

In some embodiments, the communication between the sensor and the electronic circuitry is achieved via a wired connection while in other embodiments, the communication between the sensor and the electronic circuitry is accomplished via a wireless protocol (e.g., Wi-Fi, Bluetooth, etc.).

The electronic circuitry can be configured to receive the signals from the sensor and operate on those signals to determine whether a target analyte of interest (e.g., a protein and/or an RNA or DNA segment) is present in the sample. The analysis of the signals can be achieved, for example, by software resident on the electronic circuitry.

In a related aspect, a device for collecting a biological sample is disclosed, which comprises a sample-receiving cup having a body that extends from a proximal opening to a distal opening, said proximal opening being configured for receiving a biological sample and said distal opening being configured for sealingly coupling to a sensor such at least one sensing unit of the sensor is exposed to the opening, a reservoir formed upon coupling of the sensor to the distal opening between said at least one sensing unit and a frangible membrane for storing one or more sample-processing reagents such that the sensing unit is in contact with the one or more reagents, and a shaft configured for introduction into said sample-receiving cup for piercing the frangible membrane, thereby releasing the one or more sample-processing reagents for mixing with the received biological sample.

Note that the various embodiments described above can be combined with any other embodiments described herein. The features and advantages described in the specification are not all-inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the subject matter described herein.

### BRIEF DESCRIPTION OF THE DRAWINGS

So that the present disclosure can be understood in greater detail, a more particular description may be had by reference to the features of various embodiments, some of which are illustrated in the appended drawings. The appended drawings, however, merely illustrate pertinent features of the present disclosure and are therefore not to be considered limiting, for the description may admit to other effective features as the person of skill in this art will appreciate upon reading this disclosure.

FIG. 1 schematically depicts a sample-collection device, in accordance with some embodiments.

FIG. 2 is a front-perspective exploded view of the sample-collection device, in accordance with some embodiments.

FIG. 3 is a rear-perspective exploded view of the sample-collection device, in accordance with some embodiments.

FIG. 4 schematically depicts the sample-collection device without a cap, in accordance with some embodiments.

FIG. 5 is a top perspective view of the sample-collection device, in accordance with some embodiments.

FIG. 6 is a bottom perspective view of the cap of the sample-collection device, in accordance with some embodiments.

FIG. 7A is front-perspective schematic view of the sample-collection device depicting a sample-receiving cup, a sensor, and an analysis module, in accordance with some embodiments.

FIG. 7B is a rear-perspective schematic view of the sample-collection device, in accordance with some embodiments.

FIG. 7C is another rear-perspective view of the sample-collection device, in accordance with some embodiments.

FIG. 8 is a rear-perspective schematic view of the sample-collection device illustrating a shaft extending from the cap, in accordance with some embodiments.

FIG. 9 is a detail view of a sensor and sample reservoir of the sample-collection device, in accordance with some embodiments.

FIG. 10 is a cross-sectional view showing relation of the shaft, sample reservoir, and sensor of the sample-collection device, in accordance with some embodiments.

FIG. 11A is a perspective view of one embodiment of a sample-collection device, in accordance with some embodiments.

FIG. 11B is a side view of the sample-collection device, in accordance with some embodiments.

FIG. 11C is a detail cross-sectional view of the sample reservoir and sensor of the sample-collection device, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein in order to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the embodiments described herein.

The present teachings are generally directed to a sample-collection device as well as a diagnostic device that can be employed to collect a biological sample (e.g., a saliva sample). In addition to collecting a biological sample, in some embodiments a sample collection device can further include one or more sensing units for detecting whether a target analyte of interest (e.g., a target pathogen) is present in a collected sample. Although in the following embodiments, various aspects of the present teachings are described in connection with a device for collecting saliva samples, it should be understood that the present teachings are not limited to collecting and analyzing saliva samples. For example, collection and analysis of other sample types are also contemplated. Example sample types can include, without limitation, a subject's nasal and/or nasopharyngeal swab, urine, blood (including whole blood, serum, and/or plasma), stool, spinal fluid, and/or any other liquid solution taken or derived from the subject.

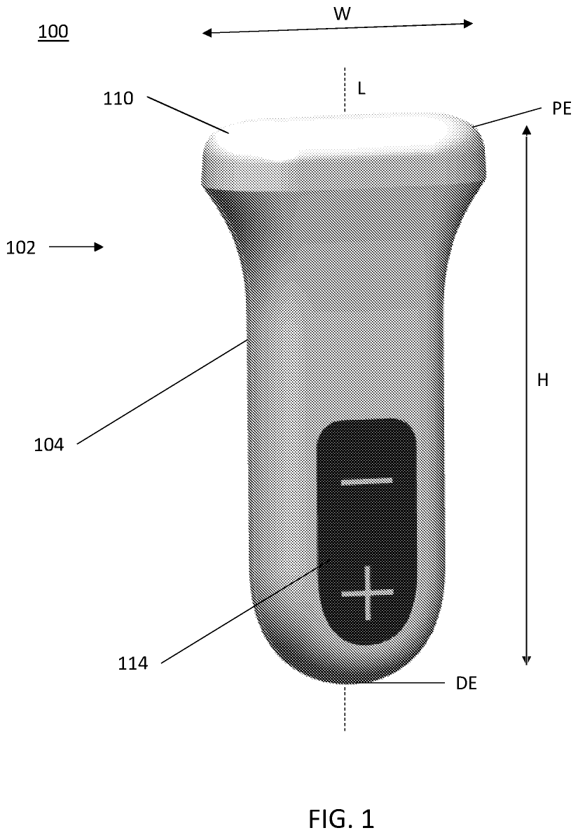

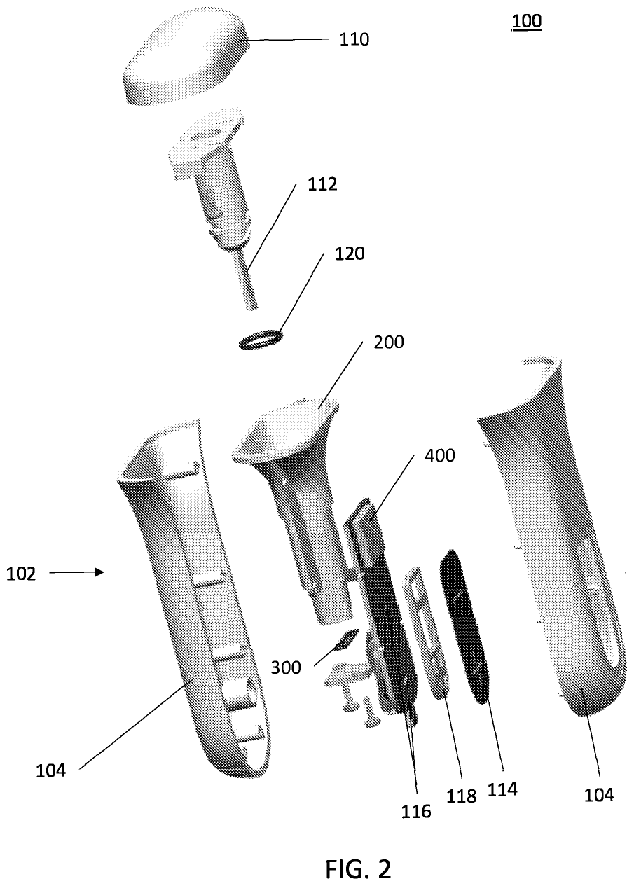

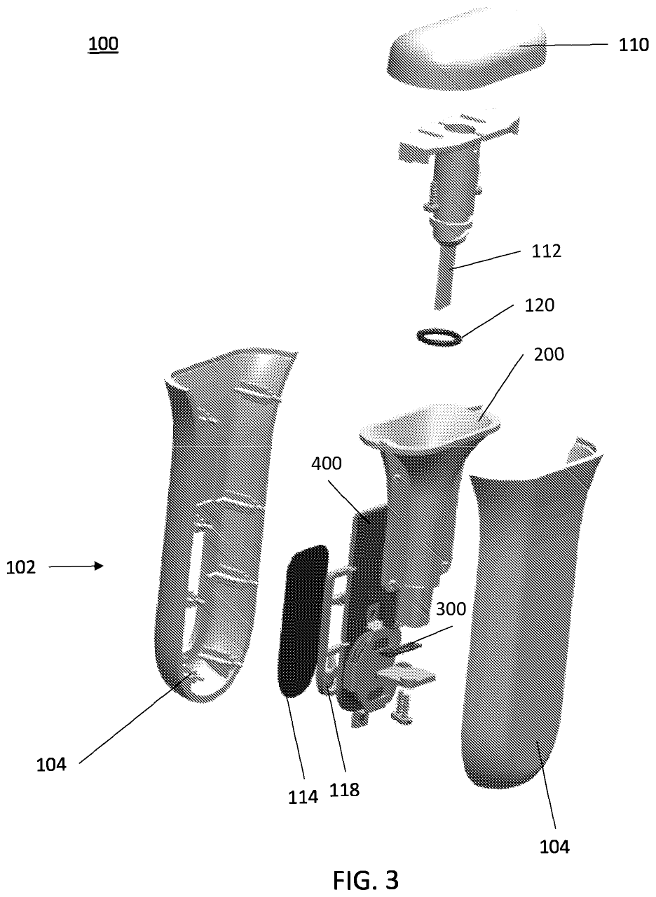

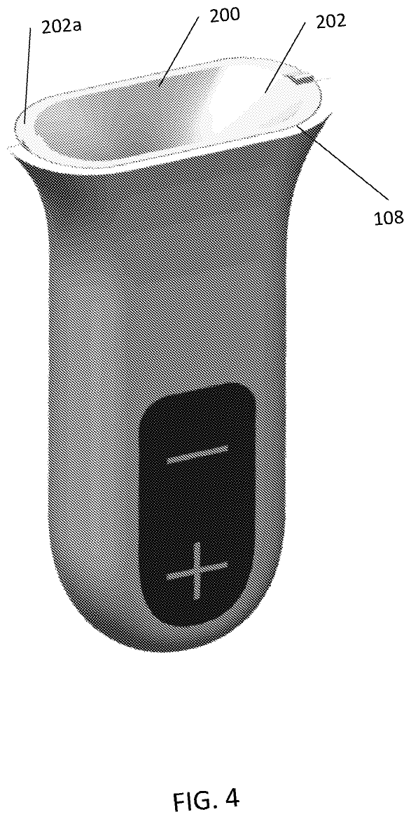

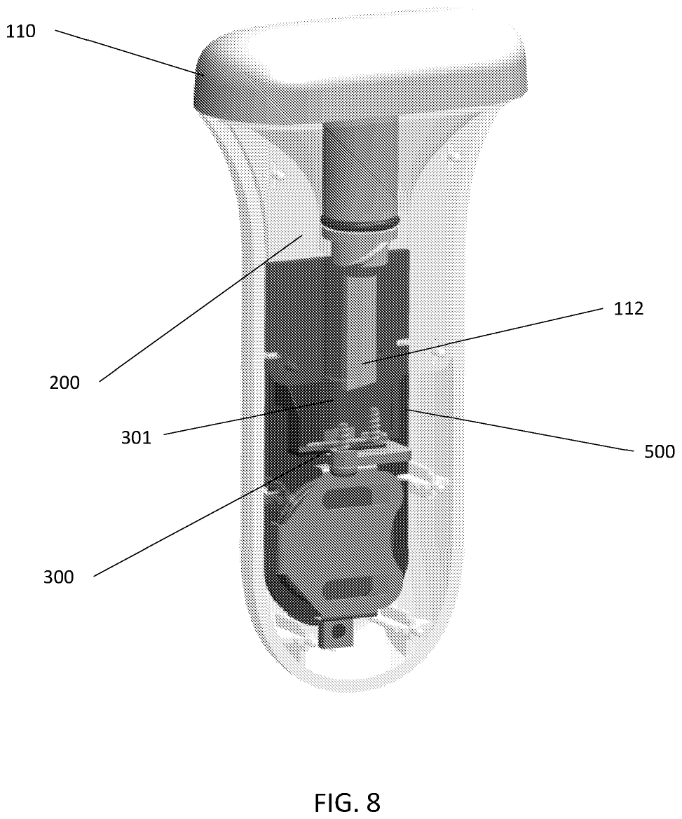

FIGS. 1-10 schematically depict a saliva collection/analysis device 100 according to an embodiment, which extends from a proximal end (PE) to a distal end (DE) and includes a hand-held housing 102 having a body 104 that surrounds an enclosure 106 (herein also referred to as a chamber 106). An opening 108 at the proximal end of the housing provides access to the enclosure 106 and can interface with a cap 110 having a shaft 112 coupled thereto. The sample collection device 100 can further include an interface 114 for communicating detection/analysis results to a user (e.g., by illuminating the illustrated positive or negative signs using a light emitting diode 116 or other illumination source in connection with a light pipe 118 or other light guide) and/or receiving inputs from a user (e.g., wherein the interface includes buttons that can be depressed by a user to turn the device on, off, initiate a test, etc.). In some embodiments, the device 100 can be coupled to a remote digital data processor, e.g., a smart phone, tablet, computer, network, etc., and can communicate results and/or receive inputs via a wired or wireless connection to the remote digital data processor. For example, in one embodiment the device 100 can be coupled to a smart phone or other external device via a Bluetooth wireless connection using a wireless communication interface included in the device.

In this embodiment, the peripheral surface of the body 104 exhibits a concave curvature along a longitudinal axis L of the device 100 for facilitating holding the collection device by a user. In some embodiments, the proximal opening 108 has a circular or elliptical profile with a diameter, e.g., in a range of about 20 mm to about 60 mm. Further, in some such embodiments, the device 100 can have an overall height H in a range of about 75 mm to about 100 mm, an overall width W in a range of about 40 mm to about 60 mm, and an overall depth or thickness in a range of about 15 mm to about 40 mm.

The saliva collection/analysis device 100 further includes a sample-receiving cup 200 having a body 201 that extends from a proximal opening 202 to a distal opening 203 and is configured for positioning in the enclosure 106. More specifically, in this embodiment, a shoulder 202a surrounding the proximal opening 202 of the fluid-receiving cup 200 can engage with the perimeter of the opening 108 at the proximal end of the housing so as to secure the fluid-receiving cup within the enclosure 106. In this embodiment, the proximal opening 202 of the sample-receiving cup is substantially circular or elliptical and its distal opening 203 has a square or rectangular profile, in other embodiments different profiles may be employed.

In this embodiment, the body of the fluid-receiving cup exhibits an inverted truncated cone-like profile in which the area of the proximal opening 202 is greater than the area of the distal opening 203.

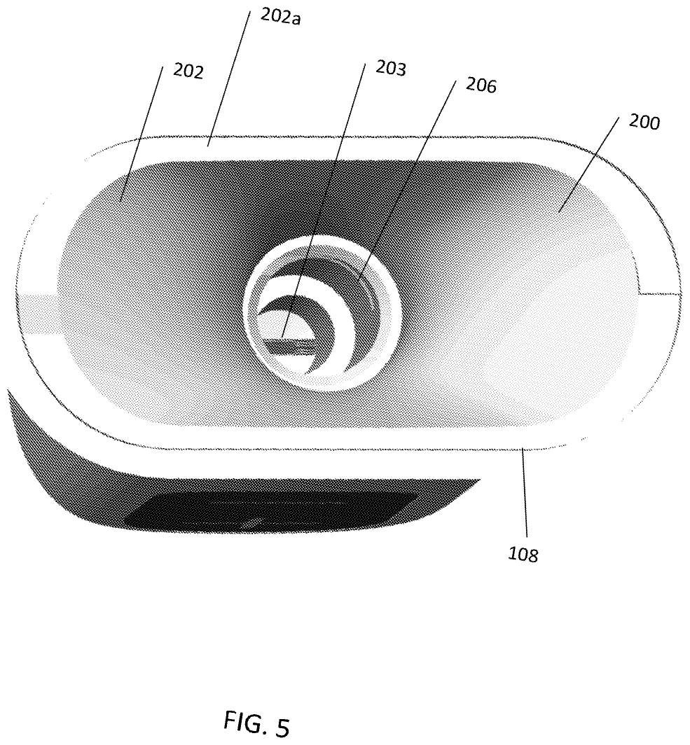

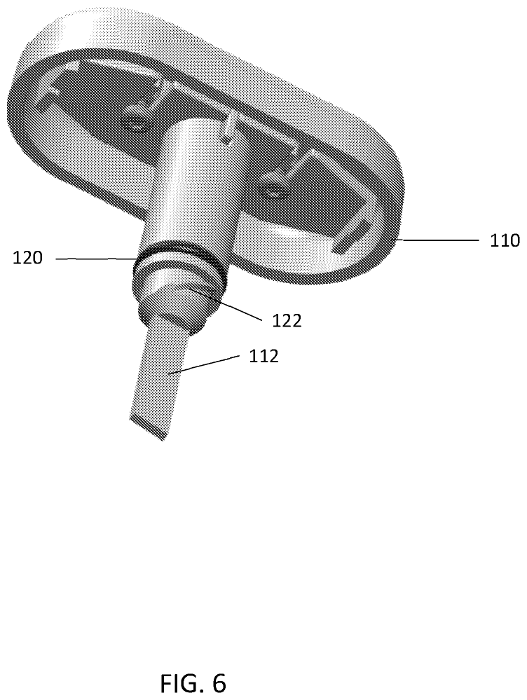

As shown in FIG. 6, the cap 110 can be coupled to a shaft 112 and can include an o-ring 120 or other sealing element to help seal the cap to the sample receiving cup 200. The shaft can also include threads 122 or other securement features to join the cap 110 and housing 102. These threads can complement threads 206 formed in the sample receiving cup 200 (see FIG. 5). In the illustrated embodiment, a quarter-turn thread 122 is utilized, but other variations are also possible.

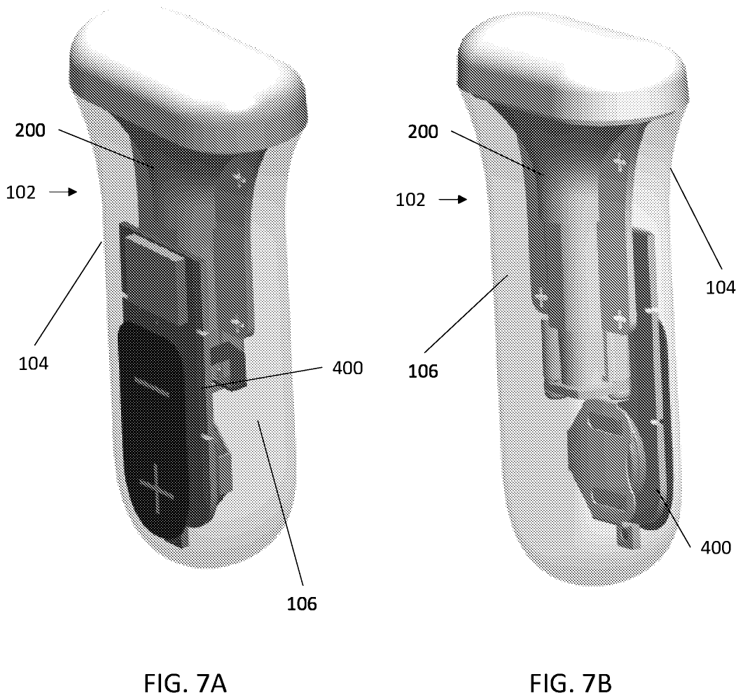

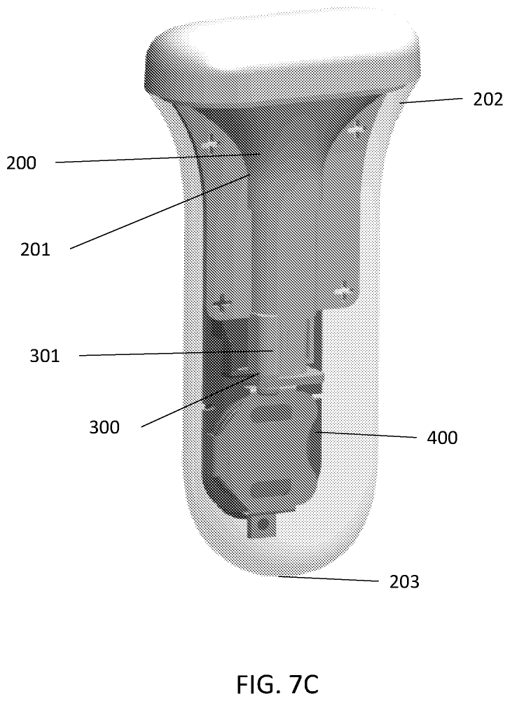

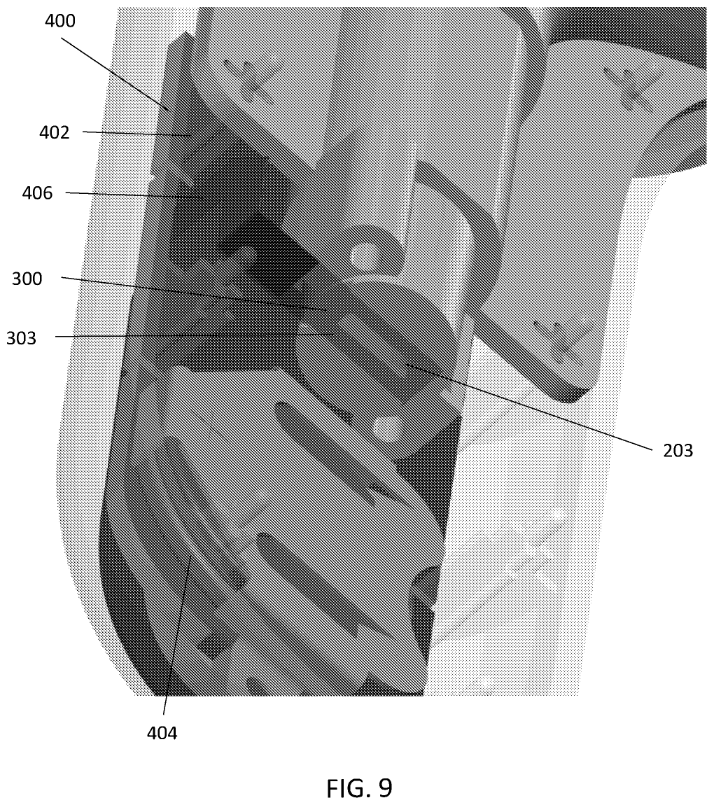

As shown schematically in FIGS. 7A-10, a sensor 300 is sealingly coupled to the distal opening 203 of the fluid-receiving cup 200 to receive at least a portion of a sample introduced into the sample-receiving cup, in a manner discussed in more detail below. More specifically, as shown schematically in FIG. 9, in this embodiment, a gasket (e.g., an O-ring, pressure-sensitive adhesive, etc.) 303 provides a fluid seal between the sensor 300 and the distal opening 203 of the fluid-receiving cup.

Further details regarding the sensor 300 and various detection methodologies that can be employed in connection with the sample collection devices disclosed herein can be found in the following patents and published applications: U.S. Pat. No. 9,664,674, entitled “Device and Method for Chemical Analysis;” US Pat. Pub. No. 2019/0079068, entitled “Device and Method for Chemical Analysis;” US Pat. Pub. No. 2019/0284615, entitled “Methods and Devices for Detection of Pathogens;” US Pat. Pub. No. 2020/0011860, entitled “Functionalized Sensor for Detection of Biomarkers;” U.S. Pat. No. 10,782,285, entitled “Device and Method for Chemical Analysis;” and US Pat. Pub. No. 2020/0300845, entitled “Methods and Devices for Detection of THC.” The entire contents of each of these publications is hereby incorporated by reference herein.

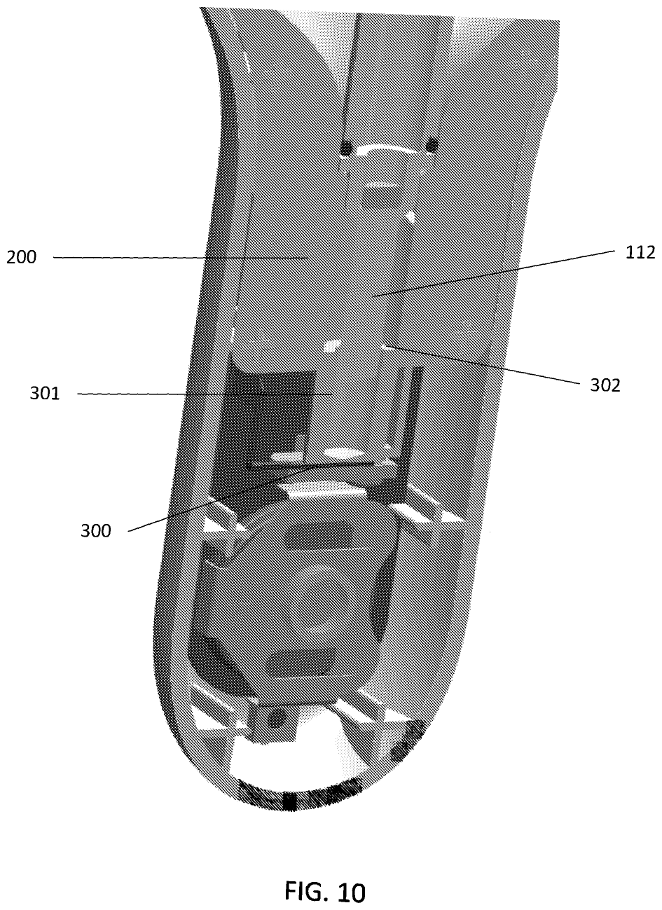

With particular reference to FIG. 10, a reservoir 301 is disposed in a distal portion of the sample-receiving cup 200. In this embodiment, a frangible membrane 302 separates the reservoir 301 from the more proximal portion, including the proximal opening 202 of the sample-receiving cup. By way of example, in some embodiments, the frangible membrane 302 can be formed of a thin polymeric material, e.g., polyurethane. In other embodiments, other types of removable partitions, such as a valve, can separate the reservoir 301 from the proximal opening 202 of the sample-receiving cup. In some embodiments, the volume of the reservoir 301 can be, for example, in a range of about 10 microliters to about 1 milliliter.

The reservoir 301 can store one or more sample-processing reagent(s) for processing a sample received in the sample-receiving cup. By way of example, the sample-processing reagent(s) can include one or more enzymes and/or any reagent capable of disrupting or lysing cell membranes, e.g., Tris-HCL (hydroxymethyl aminomethane hydrochloride), EDTA (ethylenediaminetetraacetic acid), NP-40 (nonyl phenoxypolyethoxylethanol or Tergitol-type NP-40), Triton (polyethylene glycol tert-octylphenyl ether), etc.

The sensing unit(s) of the sensor 300 are in fluid contact with the reagent(s) stored in the reservoir via the distal opening of the fluid-receiving cup 200.

As noted above, the saliva collection/analysis device 100 includes a shaft 112 (see, e.g., FIGS. 6, 8, 10) that extends from a cap 110. The shaft 112 includes a distal end (tip) that is shaped so as to allow puncturing the frangible membrane 302 upon insertion of the shaft 112 into the enclosure of the sample-receiving cup with the cap 110 engaging with the perimeter of the proximal opening of the housing, e.g., as shown in FIG. 10.

The puncture of the frangible membrane 302 results in the release of the sample-processing reagents stored in the reservoir 301 and mixing of a sample (e.g., a saliva sample) received in the sample-receiving cup 200 with the reagents released from the reservoir, thereby leading to the processing of the sample.

At least a portion of the processed sample can then come into contact with one or more sensing units of the sensor 300. The sensor can generate one or more detection signal(s), e.g., when a target analyte is present in the sample.

In this embodiment, an analysis system 400 (see, e.g., FIG. 7A) disposed in the housing 102 can receive the detection signals generated by the sensor 300 and analyze those signals, e.g., in a manner discussed in more detail below. In this embodiment and as shown in FIG. 9, the analysis system comprises a printed circuit board 402 on which electronic circuitry is disposed for providing the desired functionality of the analysis system 400. The printed circuit board 402 can be powered by batteries 404 and coupled to the sensor 300 by a ribbon cable 406 or other connecting element. In general, the analysis system 400 can be implemented in hardware, firmware, and/or software using techniques known in the art as informed by the present teachings.

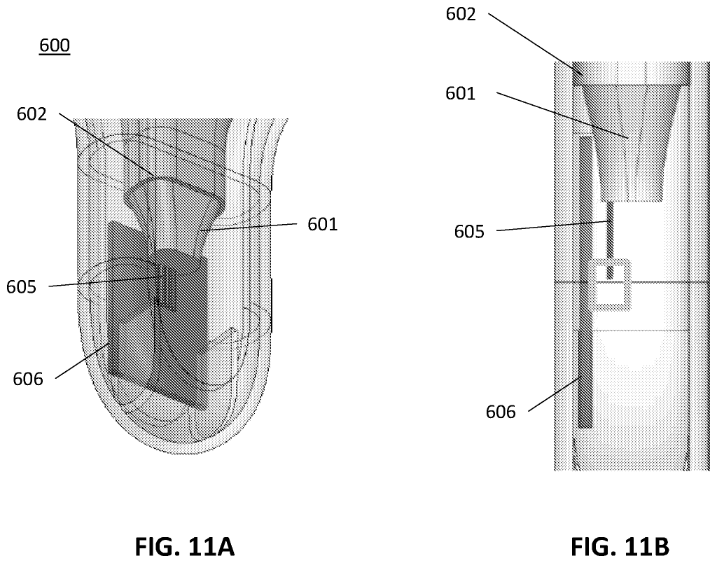

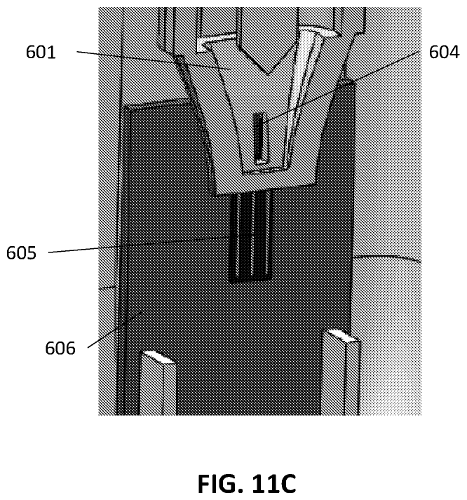

FIGS. 11A, 11B, and 11C schematically depict another embodiment of a saliva collection/analysis device 600 according to the present teachings, which is similar to the saliva collection/analysis device 100 discussed above, except that the collection/analysis device 600 includes a fluid-receiving cup 602 with a distal reservoir 601 that includes a distal opening 604 on a side surface thereof.

A sensor 605 according to the present teachings is sealingly coupled to the side distal opening 604 such that one or more sensing units of the sensor 605 will be in fluid contact with one or more reagents that are stored in the distal reservoir 601. Similar to the previous embodiment, an analysis module 606 is in communication with the sensor 605 to receive one or more detection signals generated by the sensor 605 and process those signals to determine whether one or more target analytes of interest (e.g., one or more pathogens) are present in a sample under test. The vertical orientation of the sensor 605 can, in some embodiments, allow the sensor 605 to be coupled to an analysis module 606 using a Universal Serial Bus connector on the analysis module

It will be understood that, although the terms “first,” “second,” etc., may be used herein to describe various elements, these elements should not be limited by these terms. These terms are only used to distinguish one element from another.

The terminology used herein is for the sole purpose of describing particular embodiments and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A device for analysis of a biological sample, comprising:
a sample-receiving cup having a body that extends from a proximal opening to a distal opening, said proximal opening configured for receiving a biological sample,
a sensor comprising at least one sensing unit, said sensor being sealingly connected to said distal opening of the fluid-receiving cup such that said sensing unit is exposed to said distal opening,
a reservoir formed between said at least one sensing unit and a frangible membrane for storing one or more sample-processing reagents such that said sensing unit is in contact with the one or more reagents, and
a shaft configured for introduction into said sample-receiving cup for piercing said frangible membrane, thereby releasing said one or more sample-processing reagents for mixing with the received biological sample.

2. The device of claim 1, further comprising a seal formed between said sensor and the distal opening of the fluid-receiving cup.

3. The device of claim 2, wherein said seal comprises a gasket.

4. The device of claim 1, wherein said proximal and distal openings are substantially parallel.

5. The device of claim 1, wherein said proximal and distal openings are not parallel relative to one another.

6. The device of claim 5, wherein said proximal and distal openings are substantially orthogonal relative to one another.

7. The device of claim 1, further comprising a cap from which said shaft extends, wherein the cap is configured for covering the proximal opening of the sample-receiving cup.

8. The device of claim 1, wherein said sample-receiving cup has a funnel-shaped profile such that said proximal opening has a larger area than that of the distal opening.

9. The device of claim 1, wherein said biological fluid comprises saliva.

10. The device of claim 1, wherein said one or more reagents comprises one or more enzymes.

11. The device of claim 1, further comprising a housing defining an enclosure configured for receiving said sample-collection cup.

12. The device of claim 11, wherein said enclosure comprises a proximal opening through which said fluid-collection cup can be inserted into said enclosure.

13. The device of claim 12, wherein said enclosure and said sample-receiving cup are configured such that upon insertion of said sample-receiving cup into said enclosure the proximal openings of the enclosure and the sample-receiving cup are substantially aligned.

14. The device of claim 11, wherein any of said housing and said sample-receiving cup comprises a polymeric material.

15. The device of claim 14, wherein said polymeric material comprises PDMS.

16. The device of claim 11, further comprising electronic circuitry positioned in said housing and in communication with said sensor for receiving one or more signals generated by the sensor in response to exposure of the sensor to the biological sample.

17. The device of claim 1, further comprising an interface for communicating sensor output to a user.

18. The device of claim 17, wherein the interface includes selectively illuminated indicators on an exterior surface of the device.

19. The device of claim 17, wherein the interface includes a wireless communication interface configured to connect the device to a remote digital data processor.

20. A device for collecting a biological sample, comprising:
a sample-receiving cup having a body that extends from a proximal opening to a distal opening, said proximal opening configured for receiving a biological sample and said distal opening being configured for sealingly coupling to a sensor such at least one sensing unit of the sensor are exposed to said opening,
a reservoir formed upon coupling of the sensor to the distal opening between said at least one sensing unit and a frangible membrane for storing one or more sample-processing reagents such that said sensing unit is in contact with the one or more reagents, and
a shaft configured for introduction into said sample-receiving cup for piercing said frangible membrane, thereby releasing said one or more sample-processing reagents for mixing with the received biological sample.
